SEQUENCE LISTING (17 sequences)

SEQ ID NO: 1 - DNA, 21 bp, Mus musculus
  CDS (1)...(21): 1st Polymorphic region, The 1st Polymorphism relates to the change in the 4th codon (ACN in wild type) to CCN.  Any codon is acceptable in 3rd position.
acctccaggccctcgctggag

SEQ ID NO: 2 - protein, 7 aa, Mus musculus
TSRPSLE

SEQ ID NO: 3 - DNA, 528 bp, Mus musculus
  CDS (1)...(528): 1st Polymorphic PthIh(pro) variant. Polymorphic nucleotide C in position 496, determining the presence of proline in position 166 of the PTHrP precursor.
atgctgcggaggctggttcagcagtggagtgtcctggtattcctgctcagctactccgtgccctcccgcgggcgttcggtggaggggcttggccgcaggctcaaacgcgctgtgtctgaacatcagctactgcatgacaagggcaagtccatccaagacttgcgccgccgtttcttcctccaccatctgatcgcggagatccacacagccgaaatcagagctacctcggaggtgtcccccaactccaaacctgctcccaacaccaaaaaccaccccgtgcggtttgggtcagacgatgagggcagatacctaactcaggaaaccaacaaggtggagacgtacaaagaacagccactcaagacacccgggaagaagaagaaaggcaagcctgggaaacgcagagaacaggagaaaaagaagcgaaggactcggtctgcctggccaagcacagctgcgagtggcctgcttgaggaccccctgccccacacctccaggccctcgctggagcccagcttaaggacgcattga

SEQ ID NO: 4 - protein, 175 aa, Mus musculus
MLRRLVQQWSVLVFLLSYSVPSRGRSVEGLGRRLKRAVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRREQEKKKRRTRSAWPSTAASGLLEDPLPHTSRPSLEPSLRTH

SEQ ID NO: 5 - protein, 139 aa, Mus musculus
  VARIANT (1)...(139): mature PTHrP- 1st polymorphic variant. Polymorphic aminoacid; proline in position 130.
AVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRREQEKKKRRTRSAWPSTAASGLLEDPLPHTSRPSLEPSLRTH

SEQ ID NO: 6 - DNA, 27 bp, Mus spretus
  CDS (1)...(27): 2nd polymorphic domain.  Polymorphic nucleotides; pos. n. 4  t (instead of G in w. t.), pos. 21 T (instead of G in w. t.), pos. 22 T (instead of G in w.t.)
gcttcgagtggcctgcttgattacccc

SEQ ID NO: 7 - protein, 9 aa, Mus spretus
ASSGLLDYP

SEQ ID NO: 8 - DNA, 528 bp, Mus spretus
  CDS (1)...(528): 2nd polymorphic region PthIh (SerAspTyr) variant (M. Spretus).  Polymorphic nucleotides Pos. 454, 471, 472 determining Ser in position 152, Asp in position 157, Tyr in position 158, of PTHrP precursor.
atgctgcggaggctggttcagcagtggagtgtcctggtattcctgctcagctactccgtgccctcccgcgggcgttcggtggaggggcttggccgcaggctcaaacgcgctgtgtctgaacatcagctactgcatgacaagggcaagtccatccaagacttgcgccgccgtttcttcctccaccatctgatcgcggagatccacacagccgaaatcagagctacctcggaggtgtcccccaactccaaacctgctcccaacaccaaaaaccaccccgtgcggtttgggtcagacgatgagggcagatacctaactcaggaaaccaacaaggtggagacgtacaaagaacagccactcaagacacccgggaagaagaagaaaggcaagcctgggaaacgcagagaacaggagaaaaagaagcgaaggactcggtctgcctggccaagcacagcttcgagtggcctgcttgattaccccctgccccacacctccaggacctcgctggagcccagcttaaggacgcattga

SEQ ID NO: 9 - protein, 175 aa, Mus spretus
MLRRLVQQWSVLVFLLSYSVPSRGRSVEGLGRRLKRAVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRREQEKKKRRTRSAWPSTASSGLLDYPLPHTSRTSLEPSLRTH

SEQ ID NO: 10 - protein, 139 aa, Mus spretus
  VARIANT (1)...(139): mature PTHrP-2nd polymorphic variant. Polymorphic aminoacids Ser in position 116, Asp in pos. 121, Tyr in pos. 122
AVSEHQLLHDKGKSIQDLRRRFFLHHLIAEIHTAEIRATSEVSPNSKPAPNTKNHPVRFGSDDEGRYLTQETNKVETYKEQPLKTPGKKKKGKPGKRREQEKKKRRTRSAWPSTASSGLLDYPLPHTSRTSLEPSLRTH

SEQ ID NO: 11 - DNA, 19 bp, Mus musculus
  primer
acaaagaacagccactcaa

SEQ ID NO: 12 - DNA, 19 bp, Mus musculus
  primer
acagtaccttaagctgggc

SEQ ID NO: 13 - DNA, 15 bp, Mus musculus
  primer
agcgaggtcctggag

SEQ ID NO: 14 - DNA, 15 bp, Mus musculus
  primer
ctccaggccctcgct

SEQ ID NO: 15 - DNA, 19 bp, Mus musculus
  primer
tcagcagcaccaagataca

SEQ ID NO: 16 - DNA, 19 bp, Mus musculus
  primer
ctgattcctacacaagtcc

SEQ ID NO: 17 - DNA, 18 bp, Mus musculus
  primer
aaatcctgtaacgtgtcc

(19) United States
(12) Patent Application Publication
(10) Pub. No.: US 2003/0165930 A1
(43) Pub. Date: Sep. 4, 2003

(54) Polynucleotides encoding for polymorphic isoforms of the pthrp protein, the encoded proteins and their therapeutic applications thereof

(76) Inventors: Tommaso A Dragani, Milan (IT); Giacomo Manenti, Rovato (IT)

Correspondence Address: CLARK & ELBING LLP, 101 FEDERAL STREET, BOSTON, MA, 02110, US

(21) Appl. No.: 10/276,231

(22) PCT Filed: May 11, 2001
(86) PCT No.: PCT/EP01/05413

(30) Foreign Application Priority Data
May 12, 2000 (IT) MI2000A001056

(51) Int. Cl. C12Q 1/68; G01N 33/574; A01K 67/00; C07H 21/04; C12P 21/02; C12N 5/06; C07K 14/635

(52) U.S. Cl. 435/6; 435/7.23; 435/69.1; 435/320.1; 435/325; 530/395; 536/23.5; 800/8

(57) ABSTRACT

Described herein is the identification of two polymorphic isoforms of the gene encoding the PTHrP protein, of the allelic proteins, of a first polynucleotide comprising the polymorphic domain corresponding to SEQ ID NO: 2 and comprising a first polymorphic position, of a second polynucleotide comprising a second polymorphic domain and corresponding to SEQ ID NO: 7 which encode for two variants of the PTHrP protein; also described is the use of these genes, alleles or polynucleotides to determine the risk of tumor in animals and humans and genetically modified cells and non-human animals.

### FIELD OF THE INVENTION

The present invention relates to the identification of allelic forms of the gene encoding the PTHrP protein (parathyroid hormone-related protein), to the coded proteins and to their therapeutic applications in the field of tumors.

### STATE OF THE ART

The development of tumors is a complex multi-phase process that involves molecular and cellular events still not fully known. Genetic and environmental factors are involved in regulating the tumorigenesis, as is clearly observed in experimental animal models.

With regard to the genetic component, examples have been indicated in which interaction between products encoded by specific alleles of independent genes seem to control the development of tumors.

It is therefore interesting to study the new polymorphic allelic forms correlated with pathologies in animals or in humans.

Identification of these genes and understanding their mechanism of action is particularly important in order to identify new methods for prevention and therapy of tumors.

The present invention relates to two polymorphic allelic forms of the Pthlh gene that encodes for a peptide correlated to the parathyroid hormone (PTHrP).

The Pthlh gene (parathyroid hormone-like hormone) encodes for the PTHrP peptide, a member of the parathyroid hormone family.

The PTHrP protein (parathyroid hormone-related protein) is responsible for hypercalcemia and is involved in the development of cartilage and the formation of bones and is expressed in the majority of tissues and cellular types. In contrast, the parathyroid hormone is only found in the parathyroid glands.

The PTHrP protein is a local messenger within tissues, while the parathyroid hormone has a systemic function.

Moreover, the PTHrP protein is involved in stimulating or repressing cell growth or differentiation and inhibits or stimulates the growth of specific types of cells (Strewler G. J., Mechanisms of disease, New England Journal Med., 342:177-185, 2000).

The widespread distribution of PTHrP and its processing in at least three fragments suggests the involvement of this protein in several biological functions. The three fragments are indicated respectively as “PTH-similar” amino-terminal region, “central region” and carboxy-terminal region or “osteostatin”. Osteostatin corresponds to the peptide fragment 107-139 (Strewler, 2000).

The Pthlh gene and the PTHrP protein of different animals and of humans are known in the state of the art. For example, the sequence of the Phtlh gene and the PTHrP protein of the mouse, which sometimes are referred to as wild type sequences (w.t.) are indicated in Mangin M. et al., Gene 95, (2), 195-202, 1995 (access number to the Gene Bank is NM_{—}008970) (former access number M60057.1); those of the rat in Yasuda T., et al., Mol. Endocrinol. 3, 518-525, 1989 (access number to the GenBank NM_{—}012636); those of the dog in Rosol T. J., et al., Gene 160 (2), 241-243, 1995 (access number U15593) and those of humans in Thiede M. A., et al., Proc.Natl.Acad.Sci. USA 85(13), 4605-4609, 1988 (access number GenBank NM_{—}002820).

### SUMMARY OF THE INVENTION

The authors of the present invention surprisingly found that there are allelic forms of the Pthlh gene associated with the development of tumors, in particular skin cancer and lung tumors, in animals and humans.

Therefore, a first aspect of the present invention relates to a polynucleotide comprising the nucleotide base responsible for the polymorphism of the Pthlh gene and encoding the polymorphic PTHrP protein associated with the development of tumors.

More specifically, said polynucleotide comprises the site adjacent to the polymorphic domain of the Pthlh gene (SEQ ID NO: 1) and encodes for the polymorphic PTHrP protein or a fragment thereof comprising the first polymorphic domain TSXPSLE (SEQ ID NO: 2), where X is any amino acid or may be an insertion or deletion, T (Thr) may also be I (Ile), and the polymorphic amino acid is a P (Pro) or may also be another hydrophobic amino acid.

The polynucleotide according to the invention comprises the polymorphic nucleotide corresponding to position 496 of SEQ ID NO: 3. Nonetheless, this numeric position shall obviously vary according to the sequence in various animal species and in the human species and is comprised within the sphere of this invention.

More specifically, the first polymorphism (single nucleotide polymorphism or SNP) according to the invention, corresponds to the base C and the polymorphic C of the Pthlh gene encodes an isoform of the PTHrP protein carrying the amino acid proline (Pro) in position 166 of the pre-protein (immature protein) (SEQ ID NO: 4) in place of threonine (Thr), so that both the gene and the protein in the polymorphic form according to the present invention, shall be commonly indicated with Pthlh^{Pro} and PTHrP^{Pro}, respectively. Nevertheless, the numeric position of the polymorphic nucleotide will vary according to the sequence of the different animal and human races and is comprised within the sphere of this application.

The present invention also relates to a polynucleotide encoding the PTHrP^{Pro} protein or a fragment thereof comprising the polymorphic domain of SEQ ID NO: 2, in particular the fragment indicated with the term osteostatin.

According to another aspect, the invention also relates to a polynucleotide comprising the second polymorphic amino acidic domain (seq ID NO: 7), where the polymorphic bases of the Pthlh gene correspond to positions 4, 21 and 22 of SEQ ID NO: 6. More precisely, the following bases correspond to the polymorphisms according to the invention: T (position 4), T (position 21), T (position 22) of SEQ ID NO: 6.

More specifically, said polynucleotide comprises the nucleotides adjacent to the second polymorphic domain of the Pthlh gene (SEQ ID NO: 6) and encodes the polymorphic PTHrP protein or a fragment thereof comprising the second polymorphic domain ASSGLLDYP (SEQ ID NO: 7).

The polynucleotide according to the invention comprises the polymorphic bases corresponding to positions 454, 471, 472 of SEQ ID NO: 8. Nevertheless, the numeric position will obviously vary according to the sequence of various animal and human species and is comprised within the sphere of the present invention. More specifically, the second polymorphism described in the invention corresponds to the substitution of three nucleotides (in position 454, 471 and 472) which determines the substitution of three amino acids in the PTHrP protein, which therefore brings the amino acid serine (Ser) to position 152, an aspartic acid amino acid in position 157 and a Tyr amino acid in position 158 of the pre-protein (immature protein) (SEQ ID NO: 9) in place of the amino acids Ala (152), Glu (157) and Asp (158) respectively, so that both the gene and the protein in the polymorphic form according to the present invention, shall be commonly indicated with Pthlh^{SerAspTyr} and PTHrP^{SerAspTyr} respectively. Nevertheless, the numeric position will vary according to the sequence in various animal and human species and is comprised within the sphere of the present invention.

According to another aspect, the invention relates to this polymorphic PTHrP^{SerAspTyr} protein or a fragment thereof, in particular the fragment osteostatin comprising the second polymorphic domain.

The invention also relates to oligonucleotides which hybridize with the polynucleotides comprising the Pthlh^{Pro} gene and the Pthlh^{SerAspTyr} gene, or with the gene itself or its complementary chain, and to the use of said oligonucleotides as probes to determine the presence of the Pthlh^{Pro} and Pthlh^{SerAspTyr} gene or polymorphism.

Therefore, the invention also relates to a method for identification of the polymorphisms of the Pthlh^{Pro} and Pthlh^{SerAspTyr} gene encoding respectively for the PTHrP^{Pro} and PTHrP^{SerAspTyr} protein in a subject, animal or human, comprising the step of obtaining the biological sample from the subject, and the use of these probes to identify the Pthlh^{Pro} and Pthlh^{SerAspTyr} gene or polymorphism.

The invention also relates to a kit to determine the polymorphism in a polynucleotide or in a nucleic acid sequence or in a gene encoding the PTHrP^{Pro} protein or in a fragment thereof comprising the polymorphic domain, comprising:

(a) a first container comprising the primers for PCR amplification of regions of the polynucleotide encoding the PTHrP^{Pro} protein and/or the PTHrP^{SerAspTyr} protein or their fragments thereof; and

(b) a second container comprising the PCR primers for determining said polymorphisms.

The present invention also relates to a method for the in vitro diagnosis or predisposition to the tumor, in particular skin cancer or lung tumor, comprising the step of determining the presence or absence of the Pthlh^{Pro} and Pthlh^{SerAspTyr} alleles associated with this tumor in an animal or human subject.

According to another aspect, the invention relates to a method to inhibit the expression of the Pthlh^{Pro} and Pthlh^{SerAspTyr} gene responsible for tumor development, in particular through antisense oligonucleotides or antibodies and/or peptides able to block the polymorphic proteins or fragments thereof comprising said polymorphisms, where said fragments are preferably the carboxy-terminal fragment or osteostatin.

Therefore, the invention also relates to said antisense oligonucleotides and/or antibodies and/or peptides and to pharmaceutical compositions comprising said oligonucleotides and/or antibodies and/or peptides, preferably in the presence of at least one pharmacologically acceptable excipient.

According to another aspect the invention relates to a method for the modification or transfection of animal or human cells or cell lines, with the Pthlh^{Pro} gene and/or the Pthlh^{SerAspTyr} gene or the DNA fragments carrying one or the other or both the polymorphisms and to the growth in culture of these transfected cell lines.

The cells transfected with the gene according to the invention may also be embryonic stem cells of non-human mammals, followed by implantation of said modified cells into the uterus of the adult animal to obtain transgenic non-human mammals.

According to another aspect the invention refers to a method for preparing transgenic non-human or knock-in animals, modified by the introduction of the Pthlh^{Pro} and/or Pthlh^{SerAspTyr} gene or of a polynucleotide comprising said polymorphic domains.

Preferably, the gene or DNA sequence can be introduced or associated with a tissue-specific promoter, which allows the expression of one or both polymorphic variants of the Pthlh gene in specific tissues, or which allows its activation in specific conditions.

The invention therefore relates to transgenic non-human animals obtained according to this embodiment.

### DESCRIPTION OF THE FIGURES

FIG. 1

Identification of the A→C polymorphism in the mouse Pthlh gene by allele-specific hybridization with oligonucleotides (ASO) as described in Example 2. The samples of genomic DNA of various mice strains were PCR amplified, transferred to nylon membranes and hybridized with a radioactively labelled probe represented by the oligonucleotide for the A polymorphism (Thr, w.t., left panel) or with the corresponding probe for the C polymorphism (Pro, right panel). Some examples of the presence of the A polymorphism (Thr) are A4, A5 (strain A/J); of the C polymorphism (Pro) are C7, C8 (strain C57BL/6).

FIG. 2

The morphology of the untransfected NCI-H520 cells (control) is shown in (A), of the cells transfected with Pthlh^{Thr} (w.t.) allele in (B) and of the cells transfected with Pthlh^{Pro} allele in (C).

The cells in (A) have a flat growth, the cells in (B) have a morphology similar to (A) with occasional spindle-like forms and bridge patterns between colonies, while the cells in (C) grow in clusters with the tendency of tend piling up.

FIG. 3

This figure shows the progress of in vivo tumoral growth of untransfected NCI-H520 cell (controls (●), transfected with Pthlh^{Pro} (□), and transfected with Pthlh^{Thr} (◯), inoculated in nude mice.

Data are indicated as mean volumes ±SE (Standard Error) of tumors that grow in nude mice. The graph shows that the animals inoculated with Pthlh^{Pro} transfected cells (□) have larger tumors at 8 weeks from inoculation and at this time mice were sacrificed.

FIG. 4

This Figure shows a Western Blotting experiment.

NCI-H520 cellular lines transfected with Pthlh^{Pro} (columns 5, 6, and 7) and Pthlh^{Thr} (columns 1 and 2) and untransfected (columns 3 and 4) were incubated with 1 μg of anti-PTHrP human monoclonal antibody (Ab-1, Oncogene), which reacts with residues (aa) 38-64 of the human protein and also recognizes the murine PTHrP protein. The assay indicates that the transfected Pthlh gene is capable of expressing the exogenous PTHrP protein, confirming that the different phenotype of transfected cells is due to the effect of the product of the two different Pthlh^{Pro} and Pthlh^{Thr} alleles transfected into them.

FIG. 5

The diagram shows the Kaplan-Meier estimates of survival rates of tumor bearing nude mice. Nude mice were injected twice subcutaneously (s.c.) in the left and right dorsal region with 3×10⁶ NCI-H520 cells (wt corresponding to Pthlh^{AlaGluAsp} or Pthlh^{SerAspTyr}-transfected) (10 mice/group). (Pthlh^{AlaGluAsp}=AJ32 line, Pthlh^{SerAspTyr}=Sp6 line from M. spretus SPRET/Ei. The Log-rank P=0.0113 indicates that nude mice bearing the 2^{nd} polymorphic variant transformed cells, Pthlh^{SerAspTyr}-transfected NCI-H520 cells, show a shorter survival time. The Log-rank was calculated by using the long-rank test (Peto et al., 1976, Br. J. Cancer, 35, 1-39.

### DEFINITIONS

For the purpose of this application the terms below will be interpreted as follows:

allelic variant—this is an allelic form of a known gene, distinguished from it by at least one nucleotidic base change;

polynucleotide comprising the base responsible for the polymorphism of the Pthlh gene and encoding the polymorphic PTHrP^{Pro} protein or a fragment thereof—this is any polynucleotide or nucleotide sequence that comprises the polymorphic base responsible for the polymorphism of the protein PTHrP^{Pro} or a fragment thereof;

polynucleotide comprising the base responsible for the polymorphism of the Pthlh gene and encoding the polymorphic PTHrP^{SerAspTyr} protein or a fragment thereof—this is any polynucleotide or nucleotide sequence comprising the polymorphic bases responsible for the polymorphism of the PTHrP^{SerAspTyr} protein or a fragment thereof;

oligonucleotide which hybridizes with the polynucleotide comprising the gene or with the gene itself or with its complementary chain—this is a nucleotide sequence that can be used as a probe to recognize, by hybridization, the presence of the polymorphic character;

inbred—strains of animals with all genes in homozygosis;

outbred—strains of animals without all genes in homozygosis;

knock-in animals—animals obtained by transfecting stem cells of said animal with a gene that can be activated in specific conditions (for example Shastry B. S., Molecular & Cellular Biochemistry, 181 (1-2):163-79, 1998);

transgenic animals—animals obtained by transfecting the stem cells of said animal with a gene (Hanahan D., Annual Review of Genetics, 22:479-519, 1.988);

PTHrP^{Pro}—according to the present description this is the PTHrP protein or a fragment thereof comprising the domain of SEQ ID NO: 2 comprising the 1^{st} polymorphic amino acid; this polymorphic amino acid is indicated with proline although it may also be any other hydrophobic amino acid;

Pthlh^{Pro}—this is the gene (or a fragment thereof) comprising a polymorphic base and coding for the PTHrP^{Pro} protein or a fragment thereof comprising the domain of SEQ ID NO: 2 comprising the polymorphic amino acid;

PTHrP^{SerAspTyr}—according to the present description this is the PTHrP protein or a fragment thereof comprising the domain of SEQ ID NO: 7 comprising the 2^{nd} polymorphic domain (polymorphic amino acids); these polymorphic amino acids are serin, aspartic acid and tyrosine;

Pthlh^{SerAspTyr}—this is the gene (or a fragment thereof) comprising the 2^{nd} polymorphic domain (polymorphic bases) and coding for the PTHrP^{SerAspTyr} protein or a fragment thereof comprising the nucleotide sequence of SEQ ID NO: 6 or in any case comprising the polymorphic amino acids of SEQ ID NO: 7;

SNP (single nucleotide polymorphism)—a single base responsible for the allelic form of the gene.

### DETAILED DESCRIPTION OF THE INVENTION

The authors of the present invention have found various allelic forms of the Pthlh gene responsible for polymorphisms located in the carboxy-terminal region of the PTHrP protein, and have also found that these allelic forms are associated with the development of tumors, in particular skin cancer and lung tumors, in animals and in humans.

Therefore, in a first embodiment the invention relates to a polynucleotide, gene or DNA sequence encoding for the first polymorphic domain or a fragment thereof. More specifically, the polymorphic Pthlh gene, according to the invention, encodes for the PTHrP protein or a fragment thereof comprising the first polymorphic domain: TSXPSLE (SEQ ID NO: 2), where X is any amino acid or is an insertion or deletion, T (threonine) may also be I (isoleucine), and P is the polymorphic amino acid and is a proline or may also be another hydrophobic amino acid.

In particular, the polynucleotide according to the invention (SEQ ID NO: 1), comprises any of the codons encoding for the polymorphic proline which is chosen in the group consisting of: CCT, CCC, CCA, CCG, preferably CCC. According to the degeneration of the genetic code and the preferential use of certain codons with respect to others in different organisms, the nucleotide sequence identified as SEQ ID NO: 1, may differ depending on the organism in which it is isolated or depending on the organism in which it must be expressed, although encoding for the same polymorphic domain, and is therefore included in the present invention. Therefore this invention includes all possible oligonucleotides encoding for the proteic domain defined by SEQ ID NO: 2.

The polynucleotide according to the invention thus comprises the polymorphic base C corresponding to position 496 in SEQ ID NO: 3. Nonetheless, this numeric position may vary according to the sequence of the various animal and human species and therefore the invention refers to the polymorphic base independently of its numeric position but nevertheless corresponding to the 496 position in the mouse sequence.

In particular, the Pthlh gene of the mouse C3H/He is present in the allelic form comprising the polymorphic base C in position 496 of the coding region (SEQ ID NO: 3) in place of A (adenine) present in the already known form, sometimes referred in the present description as w.t. form (Mangin et al. 1995, GenBank access number NM_{—}008970). In particular, in the allelic form found, the codon ACC encoding a Thr, in the so called w.t. form, varies in CCC encoding a Pro, and the polymorphic protein thus has the non-conservative polymorphism indicated with Thr→Pro at the position corresponding to the amino acid 166 of the precursor protein (SEQ ID NO: 4) or in position 130 of the mature protein (SEQ ID NO: 5).

As Thr is a polar amino acid while Pro is hydrophobic, the polymorphism causes a non-conservative amino acid change in the carboxy-terminal region of the Pthlh^{Pro} gene. Hence, the invention is not limited to proline, but covers all hydrophobic amino acids embodiment.

According to another embodiment, the present invention relates to a polynucleotide, gene or DNA sequence encoding for the second polymorphic domain or a fragment thereof. More specifically, the polymorphic Pthlh gene, according to this embodiment, encodes for the PTHrP protein or a fragment thereof comprising the second polymorphic domain: ASSGLLDYP (SEQ ID NO: 7). In particular, the polynucleotide according to the invention (SEQ ID NO: 6), will comprise any of the codons coding for the polymorphic amino acids, hence in particular the amino acid serine (Ser) (position 2 of SEQ ID NO: 7), the aspartic acid amino acid (Asp) (position 7 of SEQ ID NO: 7) and for the tyrosine amino acid (Tyr) (position 8 of SEQ ID NO: 7). Also included in the sphere of the present invention are the amino acid substitutions (and the corresponding nucleotidic substitutions) conservative with respect to the polymorphic amino acid. An example of conservative substitution with respect to the polymorphic amino acid of the second polymorphic domain (SEQ ID NO: 7) is the substitution of the polymorphic tyrosine in position 8 with another aromatic amino acid, for example phenylalanine (Phe) or tryptophan (Trp). At the nucleotidic level the present invention includes all those substitutions caused by degeneration of the genetic code and the preferential use of some codons in different organisms or strains. In fact, the nucleotide sequence identified as SEQ ID NO: 6, may differ according to the organism or strain of animal from which it is isolated, even though encoding for the same polymorphic domain ASSGLLDYP, and are therefore included in the present invention. Therefore the present invention includes all the possible oligonucleotides encoding for the proteic domain defined as SEQ ID NO: 7.

In particular, the polynucleotide corresponding to the second polymorphism identified according to the invention, comprises the polymorphic bases T corresponding to positions 454, 471 and 472 of SEQ ID NO: 8. Nevertheless, these numeric positions may vary according to the animal and human species and therefore the invention refers to the polymorphic base independently of its numeric position. In particular, the Pthlh gene of the mouse SPRET/Ei (M. spretus) is present in the allelic form comprising a polymorphic base T in position 454, 471 and 472 of the DNA encoding the precursor protein (SEQ ID NO: 8) in place of G (guanine) in the corresponding positions, present in the known form (Mangini et al. 1995). In particular, in the allelic form found, the codon TCG coding for serine, corresponding to position 152 of the precursor protein (seq ID NO: 9) or to position 116 of the mature protein (seq ID NO: 10) substitutes the codon GCG coding for alanine in the known allelic form; the codon GAT coding for aspartic acid in position 157 of the precursor protein (seq ID NO: 9) or 121 of the mature protein (seq ID NO: 10) substitutes the codon GAG coding for glutamic acid in the same position in the known allelic form; and the codon TAC coding for tyrosine in position 158 in the pre-mature protein (seq ID NO: 9) or 122 in the mature protein (seq ID NO: 10), substitutes the codon GAC in the same position in the known allelic form. The polymorphic PTHrP^{SerAspTyr} protein thus has the following polymorphisms indicated with Ala→Ser (pos. 152 or 116), Glu→Asp (pos. 157 or 121) and Asp→Tyr (pos. 158 or 122). Nevertheless, the invention is not limited to the amino acids indicated, but comprises all the amino acids with the same polar characteristics as the polymorphic ones.

In particular, the domains corresponding to the first and second polymorphism are present, preferably independently one another, in the proteic fragment indicated as osteostatin, which corresponds to fragment 107-139 of the mature protein in the mouse. It is therefore clear that all the other modifications of the nucleic acid leading to the production of the PTHrP^{Pro} and/or PTHrP^{SerAspTyr} protein are comprised within the sphere of the present invention.

Therefore the invention refers to any gene or portion of gene, exon, a polynucleotide, DNA sequence comprising said gene, portion of gene or exon, encoding the polymorphic PTHrP^{Pro} protein and/or coding for the polymorphic PTHrP^{SerAspTyr} protein or portions of these bearing these polymorphisms.

The invention also comprises DNA sequences encoding for proteins comprising both the polymorphic domains of SEQ ID NO: 2 and SEQ ID NO: 7.

For the sake of simplicity the genes and the polymorphic proteins described in the invention shall be indicated with Pthlh^{Pro}, Pthlh^{SerAspTyr} and PTHrP^{Pro}, PTHrP^{SerAspTyr} respectively, with the meanings set forth in the Definitions.

For simplicity, the execution and experimentation of the various aspects of the present invention were performed in the mouse animal model. Nonetheless, the invention is not limited to the mouse, but covers all mammals, animals and human, that carry the same polymorphism and which for various reasons, are not suitable for laboratory experimentation. Strains of outbred Cr—R mice (resistant to cutaneous spinocellular carcinoma) and Car-S (susceptible to cutaneous spinocellular carcinoma) were used and obtained as described in Saran et al., Carcinogenesis, Vol. 17, n. 11, 2463-2468, 1996 or in Bangrazi et al., Carcinogenesis, Vol. 11, n. 10, 1711-1719, 1990. As described in these articles, these mice are the result of appropriate cross-breeding between various inbred strains, treated according to a two phases carcinogenesis protocol with 9,10-dimethyl-1,2-benzanthracene (DMBA) and 12-O-tetradecanoyphorbol-13-acetate (TPA).

This study was performed using nude mice (with no thymus) obtained from Charles River, Calco, Italy and inbred mice obtained from Jackson Laboratories or supplied by some researchers as indicated in examples 1 and 2. It was found that the Pthlh^{Pro} polymorphism of the Pthlh gene shows a significant LD (linkage disequilibrium) with a chromosomal region of the mouse in which loci associated with the development of skin cancer and lung tumors were found.

Transfection of a human pulmonary squamous carcinoma line with mouse Pthlh^{Pro} allele produced cells with altered morphology, able to grow in clusters and piling up as found in tumoral cells, while non transfected cells and Pthlh^{Thr} transfected cells had flat (monolayer) growth.

Moreover, nude mice (with no thymus, therefore with a deficient immune systems) inoculated with Pthlh^{Pro} cells developed tumors more rapidly than those inoculated with non-transfected cells or Pthlh^{Thr} (w.t. form) transfected cells and also showed a significantly higher level of circulating calcium than the control mice.

These data confirm that both the Pthlh^{Pro} and the Pthlh^{SerAspTyr} alleles are associated with tumoral growth and with malignant hypercalcemia in the murine model.

The expression of the Pthlh^{Pro} allele and the Pthlh^{SerAspTyr} allele in a human tumoral cell line confirmed that the Pthlh^{Pro} polymorphism and the Pthlh^{SerAspTyr} polymorphism are also active on human cells. Therefore, the present invention is not limited to the mouse but covers all polynucleotides, oligonucleotides or nucleic acid sequence comprising the polymorphic variant encoding the PTHrP^{Pro} protein or coding for the PTHrP^{SerAspTyr} protein of human and non-human mammals.

In conclusion, the invention relates to all polynucleotides, oligonucleotides, nucleic acid sequences, Pthlh genes or polymorphic exons encoding the PTHrP protein or a fragment thereof, comprising the polymorphic domain TSXPSLE (SEQ ID NO: 2) and also to all polynucleotides, oligonucleotides, nucleic acid sequences, Pthlh genes or polymorphic exons encoding the PTHrP protein or a fragment thereof, comprising the polymorphic domain ASSGLLDYP (SEQ ID NO: 7). The invention also relates to all polynucleotides, oligonucleotides, nucleic acid sequences, Pthlh genes or polymorphic exons encoding the PTHrP protein or a fragment thereof, comprising the polymorphic domain TSXPSLE (SEQ ID NO: 2) together with the polymorphic domain ASSGLLDYP (SEQ ID NO: 7).

More specifically, the first polynucleotide comprises the first polymorphic position corresponding to position 496 of SEQ ID NO: 3 (mouse), and the second polynucleotide comprises the polymorphic positions 454, 471 and 472 of SEQ ID NO: 8.

The invention also relates to the PTHrP^{Pro} protein or its polymorphic fragment, indicated in SEQ ID NO: 4 or SEQ ID NO: 5 (precursor and mature protein, respectively) or in any case to a protein comprising the domain TSXPSLE (polymorphic aa underlined) (SEQ ID NO: 2).

The invention also relates to the PTHrP^{SerAspTyr} protein or its polymorphic fragment indicated in SEQ ID NO: 9 or in SEQ ID NO: 10 (precursor and mature protein, respectively) or in any case to a protein comprising the domain ASSGLLDYP (polymorphic aa underlined) (SEQ ID NO: 7).

The polymorphic allelic sequences according to the invention can also be isolated from animal or human cells.

The PTHrP^{Pro} protein and/or the PTHrP^{SerAspTyr} protein can in turn be isolated and purified starting from animal or human cells, according to prior art techniques.

As stated previously and indicated in greater detail in the experimental examples, the polymorphic genes and corresponding encoded proteins according to the invention, are associated with tumoral pathologies, in particular skin cancer and lung tumors and are implicated in malignant hypercalcemia common to various types of tumor.

Therefore, as both animal and human subjects with thess polymorphisms have or may be prone to this pathology, it is extremely important to be able to identify the polymorphisms described as important diagnostic and/or prevention tools.

In a further embodiment the invention also relates to oligonucleotides which hybridize to the polynucleotides encoding for the Pthlh^{Pro} gene or with the polynucleotide or cDNA encoding for the Pthlh^{SerAspTyr} gene or with the gene itself, or with a portion of them bearing the polymorphisms, or with their complementary chain, or mRNA.

The oligonucleotides able to recognize these polymorphic sites are used as probes to establish the presence of the Pthlh^{Pro} and/or Pthlh^{SerAspTyr} gene or polymorphism and are therefore useful in diagnosing the genetic risk to tumor development and/or to forecast its prognosis.

Hence, the invention also relates to a method for identifying the polymorphism in the Pthlh^{Pro} gene, encoding the PTHrP^{Pro} protein, and/or the Pthlh^{SerAspTyr} polymorphism, encoding the PTHrP^{SerAspTyr} protein, in an animal or a human subject, comprising the steps of obtaining the biological sample from the subject, and using the probe described to identify the Pthlh^{Pro} gene and/or the Pthlh^{SerAspTyr} gene or polymorphism.

The method according to the invention is used to diagnose the genetic predisposition to tumors or to assess their prognosis, and comprises the step of identification in an animal or human subject the presence or absence of the associated Pthlh^{Pro} and/or Pthlh^{SerAspTyr} alleles. Said tumors are preferentially skin cancers or lung carcinomas. Said method is also useful to determine the aetiology of a hypercalcemic state.

The invention also relates to a diagnostic kit for identifying and/or determining the polymorphisms of a polynucleotide or nucleic acid sequence or of the gene encoding the PTHrP^{Pro} and/or PTHrP^{SerAspTyr} protein, or a fragment thereof, comprising:

(a) a first container comprising the primers to amplify the regions of the polynucleotide encoding the PTHrP^{Pro} and/or PTHrP^{SerAspTyr} protein, or a fragment thereof;

(b) a second container comprising the primers to determine said polymorphisms or only one of these polymorphisms.

Optimal primer sequences are chosen accordingly to well established methodologies. An example of this kit and methodology is known in the literature with the term ASO and is described in Manenti G. et al., Carcinogenesis 18, 1917-1920, 1997.

The gene according to the invention or its polymorphic fragments (fragments which carries the polymorphism) are also used for the transfection of animal or human cells.

The invention therefore relates to a method for the transfection of animal or human cell lines or primary cells, with the Pthlh^{Pro} gene and/or with the Pthlh^{SerAspTyr} gene and the growth of said cells, and also to a method for the transfection of non-human animal embryonic stem cells with said genes or DNA fragments, followed by implantation of said cells in the adult animal.

The gene according to the invention or its polymorphic fragment may also be utilized to prepare transgenic non-human or knock-in animals. In particular, said transgenic non-human or knock-in animals are modified by inserting the gene under the control of a tissue-specific promoter which allows the expression of Pthlh^{Pro} and/or Pthlh^{SerAspTyr} in specific tissues or which is activated in certain conditions.

Known techniques for the preparation of transgenic (non-human) and knock-in animals are, for example, those described in Hanahan D., Annual Review of Genetics, 22:479-519, 1988 (for transgenic) and Shastry B. S., Molecular & Cellular Biochemistry, 181(1-2):163-79, 1998 (for knock-in).

Just as cells, the transformed animals (transgenic animals) are useful as research models to study the behavior and the relationships of the Pthlh gene and the PTHrP protein and their allelic variants with tumors and with calcemia levels.

As it was found that the polymorphic Pthlh^{Pro} and Pthlh^{SerAspTyr} genes and the corresponding proteins are related with the occurrence or the onset of some tumors and with malignant hypercalcemia, it is very important in therapy to block and/or inactivate this gene and/or protein.

The invention therefore also relates to antisense oligonucleotides for blocking and inactivating the Pthlh^{Pro} and/or Pthlh^{SerAspTyr} gene or their polymorphic fragments, and/or to antibodies or peptide/protein fragments to block and inactive the PTHrP^{Pro} and/or PTHrP^{SerAspTyr} protein or a polymorphic fragment therof. Therefore, a further aspect of the present invention relates to the use of these protein, protein fragments, peptides, antibodies or antisense oligonucleotides, for the preparation of pharmaceutical compositions, preferably to be used as anti-tumoral or anti-hypercalcemia drugs.

Preferably, said antisense oligonucleotide and/or antibody or peptide fragment recognize the polymorphic fragment corresponding to the osteostatin.

It is therefore also possible to prepare a pharmaceutical composition comprising said antisense oligonucleotides and/or antibodies or peptide fragments preferably in the presence of at least one acceptable pharmaceutical excipient and/or dilutant and/or carrier.

Techniques for the preparation of pharmaceutical compositions to block the stimulation of tumoral growth applicable to the invention are those indicated in EI Abdaimi K., et al., Cancer Research, 59(14):3325-8, 1999, and Falzon M., Molecular & Cellular Endocrinology, 127(1):99-108, 1997.

In order to describe the sequences included in the present invention a Sequence Listing is provided.

The present invention shall now be described according to particular embodiments in the following not limiting examples.

### EXAMPLE 1

Identification of the Polymorphic Gene Pthlh^{Pro}

3 inbred adult mice A/J, Balb/cJ and C3H/HeJ were obtained from Jackson Laboratories (Bar Harbor, Me).

The lungs were removed from these animals, the mRNA extracted according to the protocol of the Ultraspec® kit (Biotecx, Houston Tex.).

The synthesis of the corresponding full-length cDNA was obtained with MMTV RT (Gibco-BRL).

The entire region encoding the Pthlh gene of the mouse (filed in GenBank with the access number NM_{—}008970, Mangin et al.) was amplified from the lung mRNA by PCR and fragments around 200-400 bp (base pairs) in length were directly sequenced (alternatively, they were subcloned in the pCRII vector, Invitrogen, San Diego, Calif.) with ABI 377 sequencer (Perkin Elmer, Roche).

Assembly of the sequences obtained by independent PCR products allowed the creation of a consensus sequence for the various strains to be obtained. These sequences were then compared to one another. It was found that the mice A/J and Balb/cJ had the Thr (Pthlh^{Thr}) allele, while the mouse C3H/HeJ had the polymorphic Pthlh^{Pro} allele.

In conclusion, it was found that the Pthlh^{Pro} allelic variant of the Pthlh gene has the C polymorphism in position 496 (SEQ ID NO: 3), which causes a change Thr→Pro (ACC→CCC) of the amino acid 166 of the precursor protein (SEQ ID NO: 4) and in position 130 of the mature protein (SEQ ID NO: 5).

TABLE 1
Analysis of the nucleotidic polymorphism
of Pthlh in three strains of inbred mice

| Strain | Nucleotides 496 |
|---|---|
| A/J and Balb/cJ | A |
| C3H/HeJ | C |

### EXAMPLE 2

Distribution of the Pthlh^{Thr} (w.t.) and Pthlh^{Pro} Alleles in Relation to Different Mouse Strains.

75 different mouse strain, as listed in Table 2 below, were tested to check the distribution of the Pthlh^{Thr} and Pthlh^{Pro} alleles.

The mice or the genomic DNA were obtained from Jackson Laboratories (Bar Harbour, Me.) and from Dr. I. Nakashima (Nagoya University, Nagoya, Japan), (O20/A), Dr. M. Nishimura (Hamamatsu University School of Medicine, Hamamatsu, Japan) (STS/A) and Dr. M. Mandel (NCI, Bethesda, Md., United States) (NGP/N).

The presence of nucleotidic differences determining a change in the amino acid sequence was verified through allele-specific oligonucleotidic hybridization (ASO) (Manenti G., et al., 1997).

The genomic DNA of these mice was extracted from the spleen using standard methods (Genomix kit, Talent, Trieste, Italia).

The DNA fragments comprising the polymorphism were PCR amplified using the primers: 5′-ACAAAGAACAGCCACTCAA-3′ (SEQ ID NO: 11) and 5′-ACAGTACCTTAAGCTGGGC-3′ (SEQ ID NO: 12) and transferred to nylon membranes.

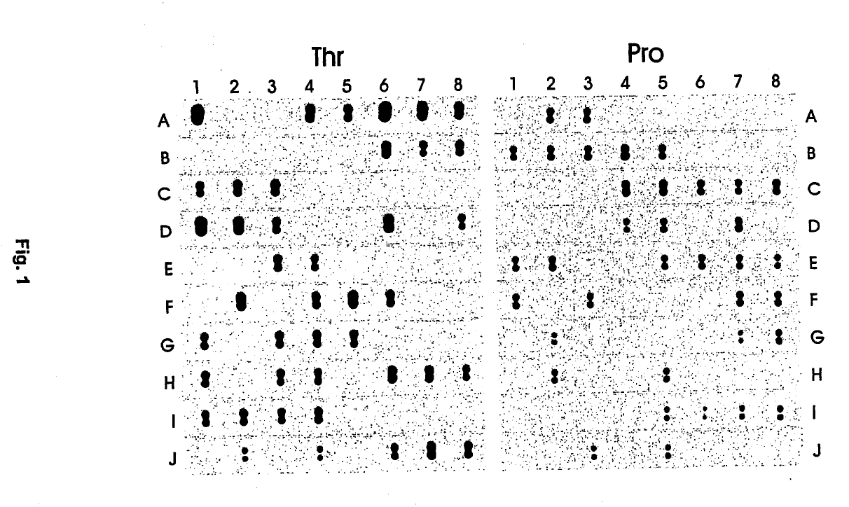

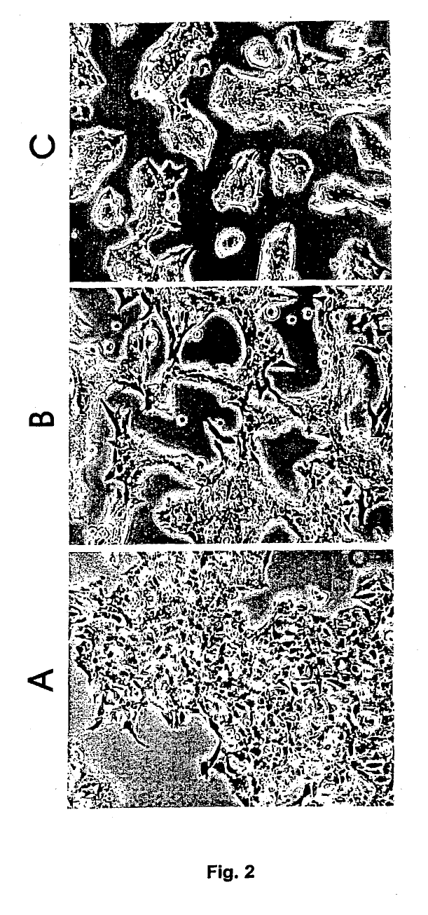

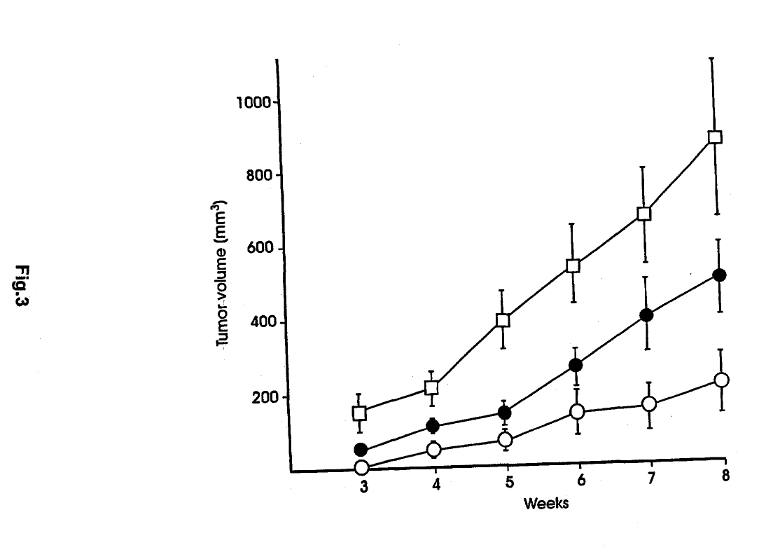

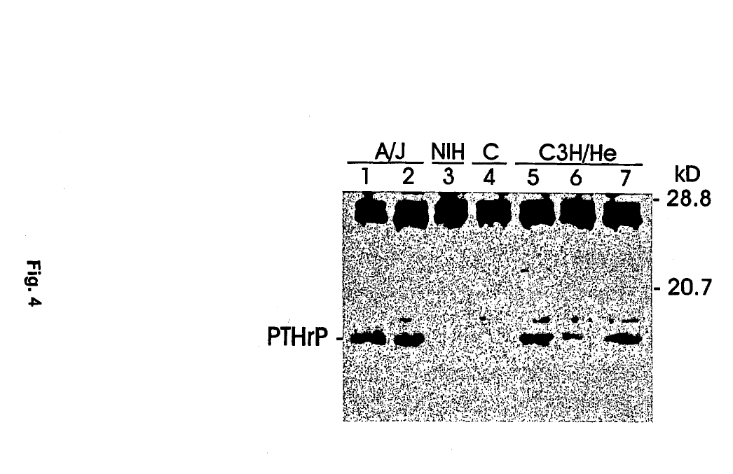

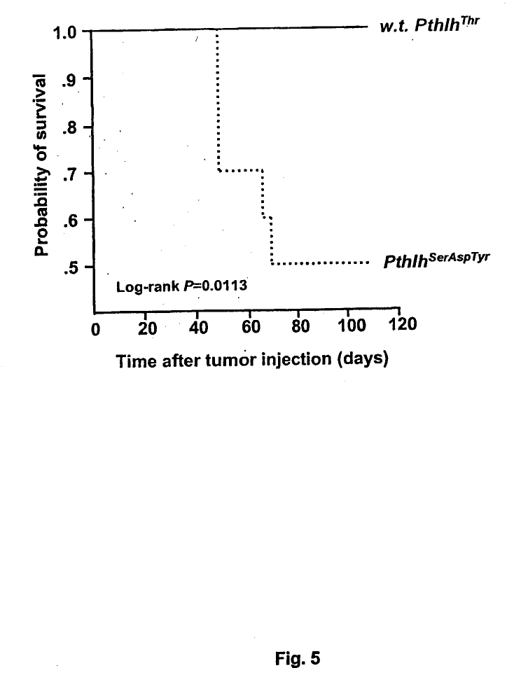

Oligonucleotides of 15 bp (15 mer) specific for the codon ACC encoding Thr (5′-AGCGAGGTCCTGGAG-3′) (SEQ ID NO: 13) and for the polymorphism CCC encoding Pro (5′-CTCCAGGCCCTCGCT-3′) (SEQ ID NO: 14) were labelled at the 5′ end with gamma ³²P-dATP and hybridized, again according to the ASO method described in Manenti as above. The results of the ASO technique regarding the results of the polymorphisms found are shown in FIG. 1.

The autoradiographic signals were measured and quantified by means of an image analysis system (Phosphorlmager, Master Image, Pharmacia). On the basis of the ratio of signals obtained, the genotype was attributed to one of the two alleles. To facilitate implementation of this experiment software was used for this allocation.

Table 2 below shows the association of the Pro or Thr polymorphism in different strains of mouse.

TABLE 2
Distribution pattern of the Pthlh^{Pro} and Pthlh^{Thr} alleles as a function of different
murine strains

| Strains | No. of strains | Pthlh allele |
|---|---|---|
| AXR/J, AU/SsJ, C3H/HeJ, C57BL/10J, C57BL/6J, | 33 | Pro |
| C57BLKS/J, C57BR/cdJ, C57L/J, C58/J, CALB/Rk, CE/J | | |
| DBA/1J, DBA/2J, IDH2/Ei, KK/HIJ, LDH2/Ei, LG/J, | | |
| MOLC/Rk, MOLD/Rk, MOLF/Ei, MOLG/Dn, NON/LtJ, | | |
| NZB/BINJ, NZO/H1J, NZW/LacJ, PL/J, SB/Le, SF/CamEi, | | |
| SJL/J, SK/CamEi, SKIVE/Ei, WB/Re, YBR/Ei | | |
| 129/J, A/J, BALB/cBy, BDP/J, BUB/BnJ, CASA/Rk, | 39 | Thr |
| CAST/Ei, CBA/CaJ, DDY/Jcl, FVB/NJ, I/LnJ, LP/J, M. | | |
| caroli, M. pahari, MA/MyJ, NGP/N, NOD/LtJ, NOR/LtJ, | | |
| O20/A, P/J, PANCEVO/Ei (M. hortulanos), PERA/Rk, | | |
| PERC/Ei, Peru Atteck/Ei, RBF/DnJ, RF/J, RIIIS/J, | | |
| SEA/GnJ, SEC/1ReJ, SI/Col, SM/, SOD1/Ei, SPRET/Ei (M. | | |
| spretus), ST/bJ, STS/A, SWR/J, TIRANO/Ei, WSB/Ei, | | |
| ZALENDE/Ei | | |

### EXAMPLE 3

Transfection of Cells with the Pthlh Gene and with its Allelic Variant; Association of the Pthlh^{Pro} Gene to the Lung Tumor and to Hypercalcemia.

Transfection of Cell Lines

The region encoding the Pthlh gene was reverse transcribed using 1 μg of total RNA extracted from the lungs of mice A/J and C3H/He, according to the protocol of the Ultraspec® kit (Biotecx, Houston Tex.).

The synthesis of the cDNA was performed with MMTV RT (Gibco-BRL) and the primer used was 5′-TCAGCAGCACCMGATACA-3′ (SEQ ID NO: 15).

Aliquots of the products of the reverse transcription (RT) reactions were PCR amplified using a forward primer positioned 40 bp upstream of the ATG codon (5′-CTGATTCCTACACMGTCC-3′) (SEQ ID NO: 16) and the reverse primer was located 41 bp downstream of the TGA stop codon (5′-AMTCCTGTAACGTGTCC-3′)(SEQ ID NO: 17).

The amplified fragments were subcloned in the eukaryotic cloning vector (TA-cloning) pCR 3.1 (Invitrogen) and placed under control of the cytomegalovirus (CMV) promoter.

The cloned sequences belonging to the two different strains were resequenced to avoid the use of clones containing any possible mutation introduced by the DNA-polymerase enzyme during the PCR reaction.

Human lung tumor cells (human lung squamous cell line) NCI-H520, obtained from American Type Culture Collections, Rockville, Md. (ATCC), were transfected with the recombinant vectors pCR 3.1, obtained above, containing mouse Pthlh^{Pro} and Pthlh^{Thr} alleles. The transfected clones were selected 2 days after transfection in selective medium containing 1 mg/ml G418 (gentamycin).

Growth of the transfected cells is shown in FIG. 2. The morphology of the non-transfected NCI-H520 cells is shown in (A), of the Pthlh^{Pro} transfected cells in (C) and of the Pthlh^{Thr} transfected cells in (B).

The NCI-H520 cells transfected with Pthlh^{Pro} grew piled up and in such way as to form clusters (C), while non-transfected cells grew flat in vitro (A). The Pthlh^{Thr} transfected cells (B) have a morphology similar to (A) with occasional spindle-like forms and bridge patterns between colonies.

These results confirm that the Pthlh^{Pro} transfected cells had undifferentiated and irregular growth, typical of tumors.

Specimen of in Vivo Tumor Growth

Nude mice (with no thymus) were obtained from Charles River.

Two groups of 20 mice were inoculated with 3×10⁶ NCI-H520 cells containing Pthlh^{Pro} or Pthlh^{Thr}, respectively, subcutaneously (s.c.) at the peritoneal level in the left and right dorsal region.

15 mice were instead inoculated with 3×10⁶ non-transfected NCI-H520 cells (controls) using the same protocol.

The diameter of the tumors developed by the mice was measured each week. Eight weeks after the beginning of the treatment, the tumors were excised, fixed in buffered formalin, embedded in paraffin, cut into sections and stained with hematoxylin and eosin.

The tumors had a morphology of poorly differentiated squamous carcinoma cells, as expected from these cellular lines, independently of the type of Pthlh allele transfected.

The in vivo growth rate of Pthlh^{Pro} transfected tumor cells was significantly faster than the non-transfected control cells (P=0.009) and the Pthlh^{Thr} transfected cells (P<0.001).

In fact, the Pthlh^{Pro} transfected cells produced large tumors. For this reason, 8 weeks after inoculation the mice were sacrificed and the experiment was terminated, as shown in FIG. 3.

These results confirm the association of the Pthlh^{Pro} allele with the proliferation of tumor cells in vivo.

The level of the electrolytes in the blood was measured in nude mice inoculated with cells transformed with the Pthlh^{Pro} allele and in the control mice, inoculated with cells transformed with the normal allele (Pthlh^{Thr}). The data obtained are shown in table 3.

TABLE 3
electrolytes levels in the blood of nude
mice inoculated with cells bearing different alleles.

| Allele | n* | Na⁺ | K⁺ | Ca⁺⁺ | Cl⁻ |
|---|---|---|---|---|---|
| Pthlh^{Thr} | 7 | 156.7 ± 0.9 | 4.77 ± 0.27 | 0.71 ± 0.08 | 112.9 ± 0.8 |
| Pthlh^{Pro} | 7 | 154.9 ± 1.2 | 5.99 ± 0.67 | 1.17 ± 0.08 | 113.9 ± 0.7 |

n*: number of mice with tumor

The data indicated in table 3 show that in the nude mice inoculated with cells transfected with the Pthlh^{Pro} allelic form, no significant differences in the hematic levels of sodium, potassium and chlorine, were observed and the levels are comparable with the control. Instead, a significant difference is observed in the level of calcium, which is substantially higher than the control. The same was done in made mice inoculated with cells transfected with the second polymorphism see table 5.

Verifying the Presence of the PTHrP^{Pro} Protein

The presence of the PTHrP^{Pro} protein in the cellular lines obtained as above was examined using Western blotting.

Protein extracts (800 μg), obtained from control cells transfected with the Pthlh alleles as above, were mixed with 1 μg of anti-human monoclonal antibody PTHrP, which reacts with the amino acid residues 38-64 of the human protein and also recognizes the murine PTHrP protein (Ab-1, Oncogene).

The experiment indicates that the transfected Pthlh gene is capable of expressing the exogenous PTHrP protein and this confirms that the different phenotype of the transfected cells is due to the effect of the two different Pthlh^{Pro} and Pthlh^{Thr} alleles introduced into it.

### EXAMPLE 4

Association of the Pthlh^{Pro} Gene with Skin Cancer

Car-S mice (susceptible to cutaneous spinocellular carcinoma) and Car-R (resistant to cutaneous spinocellular carcinoma) were obtained as in Bangrazi et al., Carcinogenesis, Vol.11, n.10, 1711-1719, 1990. These animals were treated for 13 generations (N13) with two weekly applications of 1.0 g of 12-O-tetradecanoylphorbol-13-acetate (TPA) for 4 weeks.

Two days after the last treatment, the mice were sacrificed and the skin excised and frozen.

The genomic DNA of 19 Car-R mice and 19 Car-S mice was extracted from the spleen using standard methods (Genomix kit, Talent, Trieste, Italia). Total RNA was prepared from the skin with the Ultraspec® Kit (Biotecx, Houston, Tex.).

Analysis of the polymorphism, performed with the ASO method as described in the previous examples, in the lines of mice selected phenotypically for susceptibility (S) and resistance (R) to skin cancer showed that the Pthlh^{Pro} allele was present at the level of homozygosis in 18 of the 19 Car-S mice, while the Pthlh^{Thr} allele was present in homozygosis in all 19 of the Car-R mice, as shown in Table 4.

TABLE 4
Distribution pattern of the Pthlh^{Pro} and Pthlh^{Thr} alleles
in Car-R and Car-S mice, selected phenotypically for
resistance and susceptibility, respectively, to skin cancer

| | Number of chromosomes¹ | |
|---|---|---|
| Line of outbred mouse | Pthlh^{Pro} | Pthlh^{Thr} |
| Car-R | 0 | 38 |
| Car-S | 37 | 1 |

¹log P = 19.9, Fisher's exact test

In conclusion, on the basis of the examples indicated above it was found that the amino acid polymorphism of Pthlh^{Pro} showed a significant LD (linkage disequilibrium) with susceptibility to skin cancer (−log P=19.9).

The LD between the Pthlh alleles and the predisposition to the tumor were evaluated using Fisher's exact test. Significance values were indicated by transformation into negative logarithms of P values (−log P) (Manenti G., et al., Genome Res. 9, 639-646, 1999).

### EXAMPLE 5

Identification and Characterization of the Polymorphism Pthlh^{SerAspTyr}

The Pthlh^{SerAspTyr} allele was cloned by retrotranscription of total RNA from SPRET/Ei mice essentially as described in Example 3.

cDNA synthesis and PCR fragment cloning was performed as described for the Pthlh^{Pro} and Pthlh^{Thr} alleles, but starting from cDNA of normal lung of SPRET/Ei (M. spretus) mice and using the same primers of Example 3. NCI-H520 (American Type Culture Collections, Rockville, Md.) cells were transfected with 1.5-7.5 μg of linearized DNA of recombinant pCR 3.1 expression vectors containing the sequence encoding for mouse Pthlh^{SerAspTyr} allele, corresponding to SEQ ID NO: 9, using Superfect® reagent (Quiagen). Transfected clones were selected 2 days after transfection in selective medium containing 1 mg/ml G418 (gentamycin). A clone expressing the PthrP protein (Sp6) was selected and injected s.c. into 10 nude mice.

Nude mice were injected twice subcutaneously (s.c.) in the left and right dorsal region with 3×10⁶ of either the polymorphic allele Pthlh^{SerAspTyr} or the w.t. allele Pthlh^{Thr}-transfected NCI-H520 cells. Tumor diameter was measured weekly and blood samples were collected for electrolyte analysis from mice bearing s.c. tumors. For electrolyte analysis, an additional control group of 10 nude mice received s.c. injection of 6×10⁶ (w.t.) Pthlh^{Thr}-transfected (single site) NCI-H520 cells. Blood samples were collected from mice bearing s.c. tumors and put into heparin-coated microtubes. Plasma electrolyte levels were determined by a Ciba-Corning 865 Gasanalyser. Mice were sacrificed when in poor conditions.

The in vivo growth pattern of Pthlh^{SerAspTyr} allele-transfected NCI-H520 cells was similar to that of Pthlh^{Thr}-transfected NCI-H520 cells. However, transfected Pthlh^{SerAspTyr} allele was associated with a higher mortality of tumor-bearing nuce mice (FIG. 5, P=0.0113, logrank test). Indeed, only 5/10 nude mice injected with the Pthlh^{SerAspTyr} allele-transfected NCI-H520 cells were still alive at 109 days (16 weeks) after tumor injections, whereas 10/10 nude mice injected with the Pthlh^{Thr}-transfected NCI-H520 cells were alive at the same time interval (P=0.016, Fisher's exact test).

TABLE 5
Plasma electrolyte levels in tumor-bearing nude mice

| | N^{°} of | | | | |
|---|---|---|---|---|---|
| | tumor- | | | | |
| Transfected | bearing | | | | |
| Pthlh allele¹ | mice | Na⁺ | K⁺ | Ca²⁺ | Cl⁻ |
| w.t. Thr¹⁶⁶ | 7 | 156.7 ± 0.9 | 4.77 ± 0.27 | 0.71 ± 0.08 | 112.9 ± 0.8 |
| Pro¹⁶⁶ | 7 | 154.9 ± 1.2 | 5.99 ± 0.67 | 1.17 ± 0.08² | 113.9 ± 0.7 |
| Ser¹⁵²Asp¹⁵⁷Tyr¹⁵⁸ | 8 | 156.1 ± 2.8 | 4.44 ± 0.27 | 1.76 ± 0.24² | 113.0 ± 1.5 |

¹Plasma electrolyte levels (mM) assayed in mice bearing s.c. tumors (Pthlh^{Thr}- or Pthlh^{Pro}-transfected NCl—H52O cells, 6 * 10⁶ cells/animal, single site of injection; Pthlh^{SerAspTyr}-transfected NCl—H52O cells, 6 * 10⁶ cells/animal, two sites of injection.
Background plasma electrolyte levels in four control nude mice were: Na³⁰ , 136.9 ± 4.4; K⁺, 4.81 ± 0.46; Ca²⁺, 0.86 ± 0.24; Cl⁻, 106.5 ± 5.3.
²P < 0.01, t-test analysis vs. Ala¹⁵²Glu¹⁵⁷Asp¹⁵⁸Thr¹⁸⁶ (Pthlh^{Thr},w.t.)

Electrolyte analysis in table 5 showed high calcemia levels in nude mice bearing Pthlh^{SerAspTyr} allele-transfected NCI-H520 tumor cells, as compared to nude mice bearing Pthlh^{Thr} allele-transfected NCI-H520 (Table 5, P<0.01, t-test analysis). The M. spretus-derived Pthlh^{SerAspTyr} allele displayed a cancer modifier effect in transfected human NCI-H520 tumor cells. The cancer modifier activity was associated with poor survival and high calcemia levels in tumor bearing nude mice.

1. Polynucleotide encoding a polymorphic PTHrP (ParaThyroid Hormone related Protein) or a fragment thereof, said protein or fragment comprising the polymorphic domain TSXPSLE corresponding to SEQ ID NO: 2.

2. Polynucleotide according to claim 1, comprising a polymorphic nucleotide corresponding to position 496 of SEQ ID NO: 3.

3. Polynucleotide according to claims 1-2, wherein said polymorphic domain is comprised within the region encoding for osteostatin.

4. Polynucleotide according to claims 1-3, wherein said polymorphic nucleotide is C (citosine) and wherein said polynucleotides encod a polymorphic PTHrP or a fragment thereof comprising the polymorphic domain corresponding to SEQ ID NO: 2.

5. Polynucleotide according to claim 4, wherein the polymorphic nucleotide is comprised in the codon selected in the group consisting of: CCT, CCC, CCA and CCG and encoding for the polymorphic proline of SEQ ID NO: 2.

6. Polynucleotide comprising the nucleotide sequence corresponding to SEQ ID NO: 3.

7. Polynucleotide according to claims 1-6, encoding for the protein with SEQ ID NO: 5 or a fragment thereof comprising the domain of SEQ ID NO: 2.

8. Polynucleotide encoding a polymorphic PTHrP or a fragment thereof, said protein or fragment comprising the polymorphic domain ASSGLLDYP, (SEQ ID NO: 7).

9. Polynucleotide according to claim 8, comprising the polymorphic nucleotides corresponding to positions 454, 471 and 472 of SEQ ID NO: 8.

10. Polynucleotide according to claims 8-9, wherein said polymorphic domain is comprised within the region encoding for osteostatin.

11. Polynucleotide according to claims 9-10, wherein said polynucleotide comprises the polymorphic nucleotides and encodes a polymorphic PTHrP or a fragment thereof comprising the domain corresponding to SEQ ID NO: 7.

12. Polynucleotide comprising the nucleotide sequence corresponding to SEQ ID NO: 8.

13. Polynucleotide according to claims 8-12, encoding the mature protein of SEQ ID NO: 10 or a portion thereof, comprising the domain of SEQ ID NO: 7.

14. Polynucleotide encoding a polymorphic PTHrP or a fragment thereof, comprising the polymorphic domain TSXPSLE of SEQ ID NO: 2 and the polymorphic domain ASSGLLDYP of SEQ ID NO: 7.

15. Allelic variant of the Pthlh gene encoding a polymorphic PTHrP or a fragment thereof comprising the domain of SEQ ID NO: 2

16. Allelic variant according to claim 15, encoding for the protein corresponding to SEQ ID NO: 4, or to SEQ ID NO: 5 or to polymorphic osteostain

17. Allelic variant comprising the polymorphic base corresponding to position 496 of SEQ ID NO: 3.

18. Allelic variant corresponding to SEQ ID NO: 3.

19. Allelic variant of the Pthlh gene encoding for polymorphic a PTHrP or a fragment thereof comprising the domain corresponding to SEQ ID NO: 7

20. Allelic variant according to claim 19, encoding for the protein of SEQ ID NO: 9 or SEQ ID NO: 10 or encoding a polymorphic osteostatin.

21. Allelic variant comprising the polymorphic bases corresponding to positions 454, 471, 472 of SEQ ID NO: 8.

22. Allelic variant with sequence SEQ ID NO: 8.

23. Allelic variant comprising the allelic variant according to claim 17 and according to claim 23 21.

24. Polynucleotide according to claims 1-14 or allelic variant according to claims 15-23, characterized in that it is isolated animals or from humans.

25. Polynucleotide or variant as claimed in claim 24, where said animals are mammals.

26. Polynucleotide or variant as claimed in claim 24, where said mammal is the mouse.

27. Polymorphic PTHrP or a fragment thereof comprising the polymorphic domain of SEQ ID NO: 2.

28. Protein or fragment according to claim 27, wherein the polymorphic amino acid is any hydrophobic amino acid.

29. Protein or fragment according to claims 27-28, wherein said polymorphic aminoacid is proline.

30. Protein or fragment according to claims 27-29, wherein said fragment is osteostatin.

31. Protein or fragment comprising at least one of the sequences chosen in the group consisting of: SEQ ID NO: 2, SEQ ID NO: 4, SEQ ID NO: 5, SEQ ID NO: 7, SEQ ID NO: 9, SEQ ID NO: 10.

32. Protein according to claim 31, where said fragment is osteostatin.

33. Polymorphic PTHrP or a fragment thereof comprising the polymorphic domain of SEQ ID NO: 2 and the polymorphic domain of SEQ ID NO: 7

34. Oligonucleotide able to specifically recognize by hybridisation the polymorphic domain in the polynucleotide according to claims 1-14 or in the allelic variant according to claims 15-23 or in their complementary chain.

35. Use of the oligonucleotide, as claimed in claim 34, as a probe to determine the presence of the polymorphic Pthlh gene coding for a peptide comprising the domain of SEQ ID NO: 2 and/or the domain of SEQ ID NO: 7.

36. Method for identifying the polymorphism of the Pthlh gene encoding the polymorphic PTHrP or a fragment thereof comprising the domain of SEQ ID NO: 2 and/or the domain of SEQ ID NO: 7, in an animal or human subject, comprising the steps of:
obtaining a biological sample from the subject
analyze the sample to identify said polymorphic Pthlh allelic variant.

37. Kit for the identifying and/or determining the polymorphism of a polynucleotide or nucleic acid sequence or of the gene encoding the PTHrP protein or a fragment thereof comprising the polymorphic domain corresponding to SEQ ID NO: 2 and/or the domain corresponding to SEQ ID NO: 7, comprising:
(a) a first container comprising the primers for amplification of regions of the polynucleotide encoding a polymorphic PTHrP protein or a fragment thereof comprising at least one of said polymorphic domains; and
(b) a second container comprising the oligonucleotidic primers to determine at least one of said polymorphisms.

38. Method for the in vitro diagnosis of the predisposition to the development of tumor and/or malignant hypercalcemia and/or evaluation of their prognosis, comprising the identification in an animal or human subject of the presence or absence of the Pthlh-allelic variant as defined in claims 15-23, associated to said tumor or hypercalcemia.

39. Method for the in vitro diagnosis of the predisposition to the development of tumor and/or malignant hypercalcemia and/or evaluation of their prognosis, comprising the identification in an animal or human subject of the presence or absence of the PTHrP protein or a fragment thereof as defined in claims 27-33, associated to said tumor or hypercalcemia.

40. Method according to claims 38-39, wherein said tumor is a skin cancer or lung carcinoma.

41. Method for the transfection of animal or human cells with the polynucleotide according to claims 1-14 or the variant as claimed in claims 15-23, and the growth in culture of said cells.

42. Method for the transfection of non-human animal embryonic stem cells with the polynucleotide according to claims 1-14 or with the variant according to claims 15-23, and implantation of said cells.

43. Method as claimed in claims 41-42, wherein said cells are transfected by means of the introduction of a tissue-specific promoter.

44. Isolated animal or human cell genetically transformed with the polynucleotide according to claims 1-14 or with the variant according to claims 15-23.

45. Isolated non-human animal embryonic stem cells transfected with the polynucleotide according to claims 1-14 or with the allelic variant as claimed in claims 15-23.

46. Cell cultures or isolated cells as claimed in claims 44-45, characterized in that they are additionally modified with a tissue-specific promoter.

47. Method for preparing transgenic non-human animals modified with the insertion of the polynucleotide as claimed in claims 1-14 or of the allelic variant of the gene as claimed in claims 15-23.

48. Method according to claim 47, wherein said transgenic non-human animal is modified with a tissue-specific promoter and expresses the Pthlh gene, encoding for a peptide comprising the domain of SEQ ID NO: 2 and/or of SEQ ID NO: 7, in specific tissues.

49. Method to prepare a non-human animal according to claim 48, comprising the step of transfecting the stem cells of said animal, and wherein said gene, allele or polynucleotide, is activated in the adult animal.

50. Transgenic non-human animals obtained with the method as claimed in claims 48-49.

51. Animal obtained according to the method of claim 49, characterized in that it is a knock-in animal.

52. Antisense oligonucleotide for blocking and inactivating specifically the polymorphic Pthlh gene or the DNA sequence encoding for a peptide comprising the polymorphic domain of SEQ ID NO: 2 and/or SEQ ID NO: 7, with the provision that said antisense olygonucleotide has not the following sequence: 5′TGAACCAGCGACCGCAAGCM.

53. Oligonucleotide as claimed in claims 34 and 52 for use as a therapeutic.

54. Use of the oligonucleotides as claimed in claim 53 for the preparation of anti-tumor drugs.

55. Use of the oligonucleotides as claimed in claim 53 for the preparation of a medication for the treatment of malignant hypercalcemia.

56. Antibodies or peptide fragments for blocking and inactivating specifically the polymorphic PTHrP or a fragment thereof comprising the polymorphic domain SEQ ID NO: 2 and/or of SEQ ID NO: 7.

57. Antibodies or peptide fragments according to claim 56, wherein said polymorphic fragment is osteostatin.

58. Antibodies or peptide fragments according to claim 56 for use as therapeutic.

59. Use of antibodies or peptide fragments according to claim 58 for the preparation of an anti-tumor drug.

60. Use of the antibodies or peptide fragments according to claim 58 for the preparation of a medicament for the treatment of malignant hypercalcemia.

61. Pharmaceutical composition comprising antisense oligonucleotides according to claims 52-53 and/or antibodies or peptide according to claims 56-58 and at least one acceptable pharmaceutical excipient.
